(19) United States
(12) Patent Application Publication
(10) Pub. No.: US 2019/0099154 A1
(43) Pub. Date: Apr. 4, 2019

(54) ULTRASOUND DIAGNOSTIC SYSTEM

(71) Applicant: SOCIONEXT INC., Yokohama-shi (JP)

(72) Inventors: Naoto ADACHI, Yokohama (JP); Masahiko GOTO, Yokohama (JP); Mari KOBAYASHI, Yokohama (JP); Masaya TAMAMURA, Yokohama (JP)

(21) Appl. No.: 16/131,983

(22) Filed: Sep. 14, 2018

(30) Foreign Application Priority Data
Sep. 29, 2017 (JP) 2017-189839

(51) Int. Cl. A61B 8/08; A61B 8/00; A61B 8/14; A61B 17/34

(52) U.S. Cl. CPC A61B 8/085; A61B 8/4461; A61B 8/54; A61B 2017/3413; A61B 8/463; A61B 8/145; A61B 17/3403; A61B 8/4494

(57) ABSTRACT

An ultrasound diagnostic system includes: an ultrasonic probe having a plurality of transducers and configured to obtain a plurality of pieces of ultrasound image data of a subject, the transducers being arranged in a first direction and configured to transmit ultrasonic waves toward the subject and receive ultrasonic waves reflected from the subject; a moving mechanism configured to move the ultrasonic probe in the first direction and in a second direction; and a controller including a processor configured to perform a process including, causing the moving mechanism to move the ultrasonic probe based on a position of a blood vessel in an ultrasound image, and generating blood vessel map data indicating information related to the blood vessel by referring to an image data storage in which the plurality of pieces of ultrasound image data are stored in association with respective amounts of movement in the first and second direction.

### CROSS-REFERENCE TO RELATED APPLICATIONS

This application is based on and claims priority to Japanese Patent Application No. 2017-189839 filed on Sep. 29, 2017, the entire contents of which are incorporated herein by reference.

### BACKGROUND OF THE INVENTION

### 1. Field of the Invention

The disclosures herein generally relate to an ultrasound diagnostic system.

### 2. Description of the Related Art

In recent hemodialysis treatment, a patient is provided with vascular access serving as an inlet/outlet port for removing and returning blood of the patient such that the blood of the patient is circulated through a dialyzer. In such hemodialysis treatment, properly managing and maintaining the vascular access is important for long-term treatment.

The vascular access may be sometimes managed by using a vascular access map created by a medical professional. The vascular access map is, for example, data indicating ultrasound images of a blood vessel obtained by scanning the patient's body surface (such as the upper arm) with an ultrasonic apparatus and also indicating information related to the shape, the depth from the body surface, and conditions of the blood vessel estimated by the medical professional based on the obtained ultrasound images.

In a conventional method of creating a vascular access map, it is required to acquire techniques such as scanning the body surface with the ultrasonic apparatus and estimating the shape, the depth from the body surface, and conditions of the blood vessel. In order to acquire such techniques, long experience is required. Therefore, the accuracy of a conventional vascular access map varies depending on the degree of techniques acquired by a medical professional who creates the vascular access map.

In view of the above, it is an object of the disclosed technique to readily create a highly accurate vascular access map.

### RELATED-ART DOCUMENT

### Patent Document

[Patent Document 1] Japanese Laid-open Patent Publication No. 10-33538
[Patent Document 2] Japanese Laid-open Patent Publication No. 2002-336253

### SUMMARY OF THE INVENTION

According to an embodiment, an ultrasound diagnostic system includes: an ultrasonic probe having a plurality of transducers and configured to obtain a plurality of pieces of ultrasound image data of a subject, the plurality of transducers being arranged in a first direction and configured to transmit ultrasonic waves toward the subject and receive ultrasonic waves reflected from the subject; a moving mechanism configured to move the ultrasonic probe in the first direction and in a second direction perpendicular to the first direction; and a controller including a processor being configured to perform a process including, causing the moving mechanism to move the ultrasonic probe based on a position of an image of a blood vessel to be observed of the subject in an ultrasound image indicated by a corresponding piece of the plurality of ultrasound image data, and generating blood vessel map data indicating information related to the blood vessel to be observed by referring to an image data storage in which the plurality of pieces of ultrasound image data are stored in association with respective amounts of movement in the first direction and in the second direction.

Other objects and further features of the present invention will be apparent from the following detailed description when read in conjunction with the accompanying drawings.

### BRIEF DESCRIPTION OF THE DRAWINGS

FIG. 1 is a drawing illustrating an example of an ultrasound diagnostic system according to a first embodiment:

FIG. 2 is a drawing that depicts a moving mechanism according to the first embodiment;

FIG. 3 is a drawing that depicts a probe according to the first embodiment;

FIG. 4 is a drawing illustrating functions of a controller according to the first embodiment;

FIG. 5 is a drawing illustrating an example of an image data storage unit according to the first embodiment;

FIG. 6 is a drawing illustrating an example of a map storage unit according to the first embodiment;

FIG. 7 illustrates an example of ultrasound image data obtained by an image obtaining unit;

FIG. 8 is a flowchart illustrating a process performed by the controller according to the first embodiment;

FIG. 9 is a drawing illustrating an example of a vascular access map according to the first embodiment;

FIG. 10 is a drawing illustrating an example of an ultrasound diagnostic system according to a second embodiment;

FIG. 11 is a drawing illustrating functions of a controller according to the second embodiment;

FIG. 12 is a drawing illustrating an example of a map storage unit according to the second embodiment;

FIG. 13 is a flowchart illustrating a process performed by the controller according to the second embodiment;

FIG. 14 is a drawing that depicts a method of specifying a puncture position on a vascular access map;

FIGS. 15A and 15B are drawings illustrating examples of images of a region of a patient;

FIGS. 16A and 16B are drawings illustrating a first variation; and

FIGS. 17A through 17C are drawings illustrating a second variation.

### DESCRIPTION OF THE PREFERRED EMBODIMENTS

In the following, embodiments of the present invention will be described with reference to the accompanying drawings.

### First Embodiment

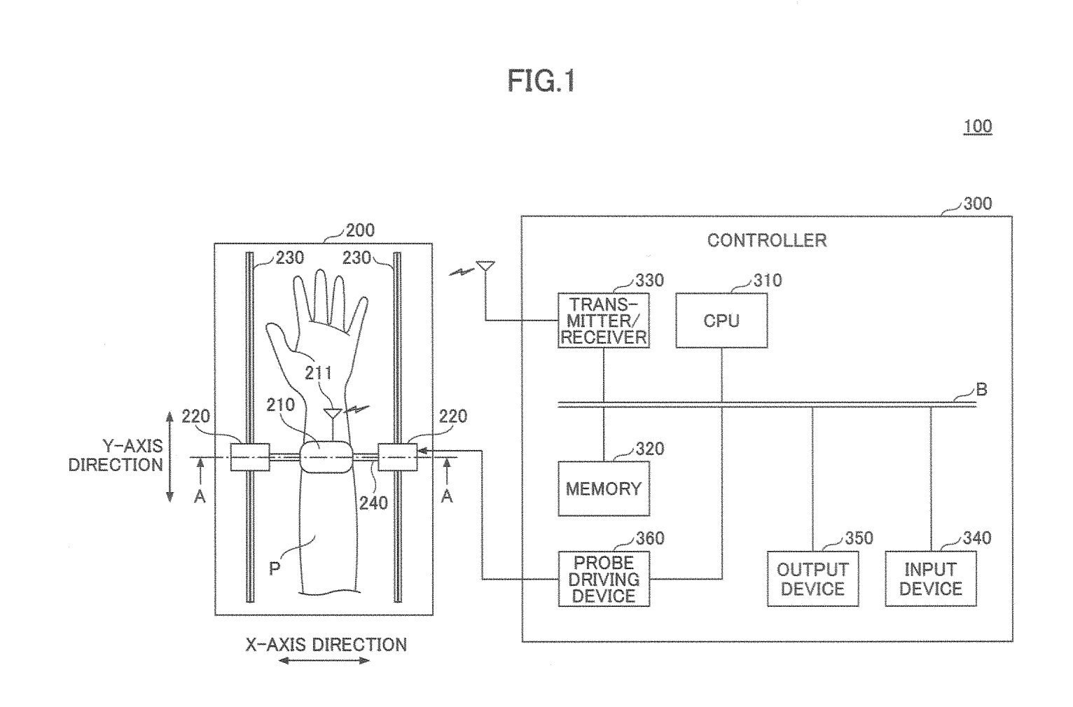

In the following, a first embodiment will be described with reference to the drawings. FIG. 1 is a drawing illustrating an example of an ultrasound diagnostic system according to the first embodiment.

An ultrasound diagnostic system 100 according to the present embodiment includes a probe moving device 200 and a controller 300.

The probe moving device 200 according to the present embodiment includes a probe 210, a moving mechanism 220, and rails 230 and 240.

The probe 210 is an ultrasonic probe that includes a plurality of transducers configured to transmit ultrasonic waves to an object and receive reflected waves from the object, and that outputs ultrasound image data indicating an ultrasound image of the object based on the reflected waves. Further, the probe 210 includes a transmitter 211 configured to transmit the ultrasound image data to the controller 300.

The moving mechanism 220 moves the probe 210 in two directions of an X-axis direction and a Y-axis direction based on an instruction from the controller 300. The moving mechanism 220 will be described later in detail.

The X-axis direction (a first direction) according to the present embodiment refers to a direction in which the plurality of transducers is arranged in the probe 210. In other words, the X-axis direction refers to a short-axis direction of the probe 210. Further, the y-axis direction (a second direction) according to the present embodiment refers to a direction perpendicular to the X-axis direction. In other words, the Y-axis direction refers to a long-axis direction of the probe 210.

The rails 230 guide movement of the moving mechanism 220 in the Y-axis direction. The rail 240 guides movement of the moving mechanism 220 in the X-axis direction.

The controller 300 according to the present embodiment is an information processing apparatus that includes a CPU (central processing unit) 310, a memory 320, a transmitter/receiver 330, an input device 340, an output device 350, and a probe driving device 360.

The CPU 310 controls the entire operation of the controller 300. To be more specific, the CPU 310 controls an amount of movement of the probe 210 in the X-axis direction and in the Y-axis direction of the probe moving device 200, for example. Further, the CPU 310 creates and outputs a vascular access map based on ultrasound image data received from the probe 210.

The memory 320 stores programs that operate the controller 300 and various calculation results performed by the CPU 310. Also, the memory 320 stores ultrasound image data received from the probe 210.

The transmitter/receiver 330 communicates with the probe moving device 200. To be more specific, the transmitter/receiver 330 receives ultrasound image data transmitted from the probe 210 and transmits information indicating an amount of movement of the probe 210 to the moving mechanism 220.

The input device 340 is used to input various types of information in the controller 300. To be more specific, the input device 340 may be a device such as a keyboard or a mouse, for example.

The output device 350 may be a display device such as a display, for example. The output device 350 according to the present embodiment displays ultrasound image data received by the transmitter/receiver 330 and a vascular access map created by the CPU 310, for example.

In a case where the controller 300 according to the present embodiment is a tablet-type information processing apparatus, the controller 300 may have a display operation device, instead of the input device 340 and the output device 350. The display operation device may be a touch panel, for example.

The probe driving device 360 instructs the moving mechanism 220 to drive. To be more specific, the probe driving device 360 transmits, to the moving mechanism 220, an amount of movement of the probe 210 in the X-axis direction and in the Y-axis direction, as calculated by the CPU 310.

In the ultrasound diagnostic system 100 according to the present embodiment, a patient's upper arm (a subject) is placed between the rails 230 of the probe moving device 200. The probe 210 obtains ultrasound image data of the upper arm and transmits the obtained ultrasound image data to the controller 300.

In the probe moving device 200 according to the present embodiment, the probe 210 obtains ultrasound image data and sends the obtained ultrasound image data to the controller 300 each time the probe 210 moves along the rails 230 and the rail 240.

Based on the ultrasound image data sequentially obtained in accordance with the movement of the probe 210, the controller 300 according to the present embodiment calculates an amount of movement of the probe 210 in the X-axis direction such that an image of the blood vessel to be observed is located at the center of the ultrasound image data obtained by the probe 210. Subsequently, the controller 300 causes the moving mechanism 220 to move the probe 210 in the X-axis direction in accordance of the calculated amount of movement and also causes the moving mechanism 220 to move the probe 210 in the Y-axis direction.

In the present embodiment, the probe 210 is moved in the X-axis direction in accordance with the calculated amount of movement and in the Y-axis direction. Thus, the probe 210 can meander in accordance with the shape of the blood vessel to be observed. Further, in the present embodiment, the probe 210 is moved such that the blood vessel to be observed is always located at the center. Accordingly, it is possible to create a vascular access map by using ultrasound image data having the target blood vessel always located at the center.

Accordingly, in the present embodiment, it becomes unnecessary for a medical professional to scan a patient's body surface by placing the probe 210 on the body surface while visually finding a blood vessel to be observed from ultrasound images.

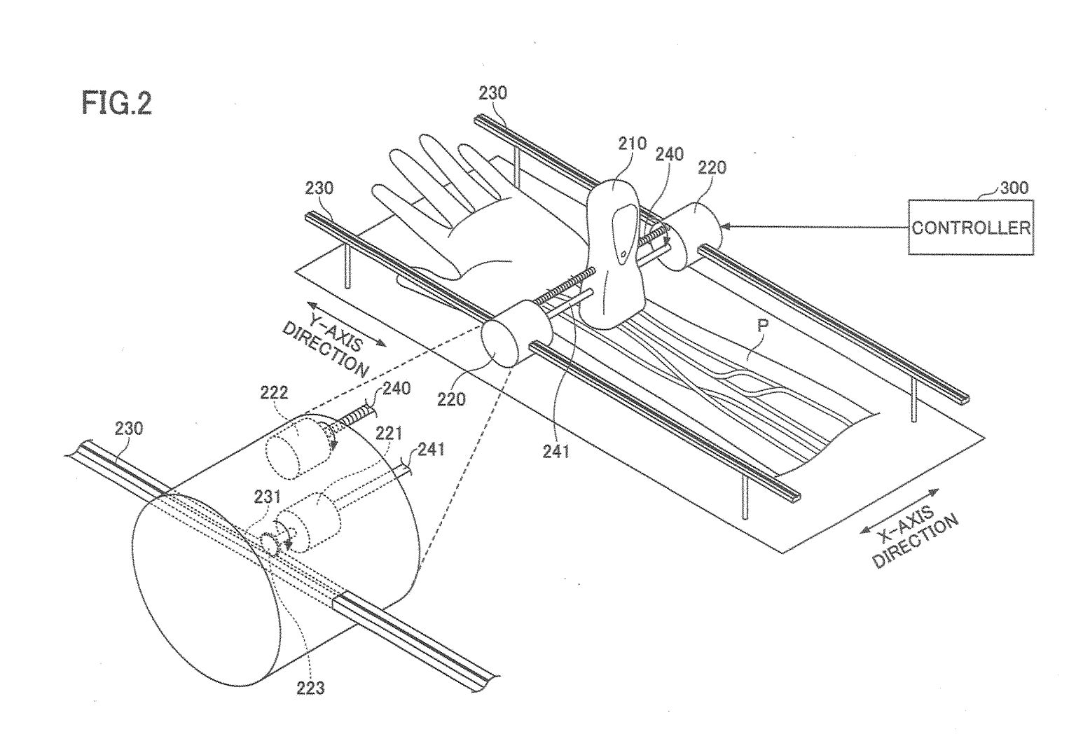

In the following, referring to FIG. 2, the moving mechanism 220 of the probe moving device 200 according to the present embodiment will be described.

FIG. 2 is a drawing that depicts the moving mechanism according to the first embodiment. The moving mechanism 220 according to the present embodiment includes motors 221 and motors 222. The motors 221 are moving means for moving the probe 210 along the rails 230. In other words, the motors 221 are moving means for moving the probe 210 in the Y-axis direction.

The motors 222 are moving means for moving the probe 210 along the rail 240. In other words, the motors 222 are moving means for moving the probe 210 along the X-axis direction.

In the moving mechanism 220, gears 223 provided on both ends of a rotating shaft 241 of the motors 221 mesh with teeth formed on each of the rails 230. In the moving mechanism 220, the gears 223 rotate in accordance with the rotation of the rotating shaft 241, and the probe 210 moves in the Y-axis direction in accordance with the rotation of the gears 223. Therefore, a direction in which the probe 210 moves along the Y-axis is determined by the rotation direction of the rotating shaft 241 (the motors 221).

Further, the rotating shaft 241 according to the first embodiment penetrates a housing of the probe 210, and supports the probe 210 such that the transducers, which will be described later, face the body surface of the patient all the time.

In the moving mechanism 220, the motors 222 cause the rail 240 to rotate. The rail 240 is a screw shaft. The rail 240 forms a ball screw mechanism, together with a nut and balls provided in the housing of the probe 210. Therefore, a direction in which the probe 210 moves along the X-axis is determined by the rotation direction of the rail 240 shaft (the motors 222).

The moving mechanism 220 according to the present embodiment is provided with a receiving unit that receives, from the probe driving device 360 of the controller 300, a signal indicating an amount of movement in the X-axis direction and in the Y-axis direction, and is also provided with a driving unit that rotates the motors 221 and the motors 222 in accordance with the amount of movement.

Further, in the above-described configuration, the moving mechanism 220 according to the present embodiment uses the motors 221 and the motors 222 to move the probe 210 in the Y-axis direction and the X-axis direction, respectively. However, the present invention is not limited to this configuration, as long as the moving mechanism 220 can independently move the probe 210 in the X-axis direction and in Y-axis direction. Therefore, the moving mechanism 220 may be a mechanism that moves the probe 210 in each of the X-axis direction and the Y-axis direction by pushing or pulling the probe 210, for example.

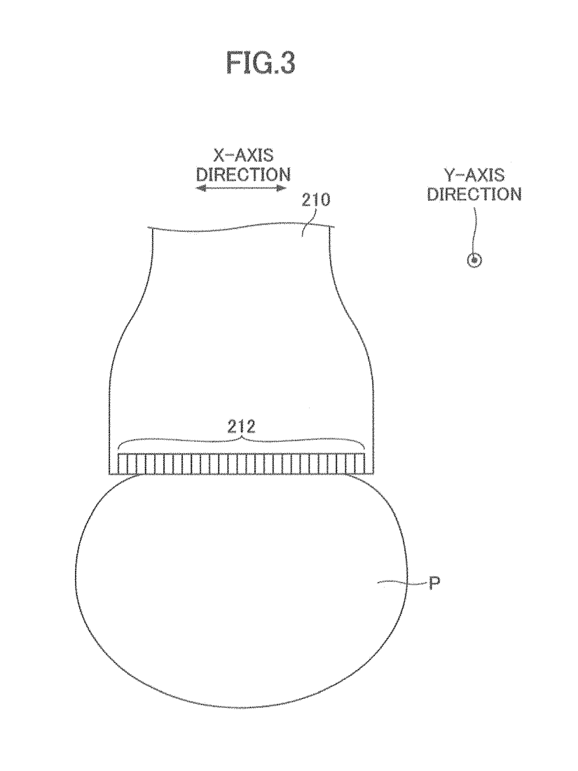

FIG. 3 is a drawing that depicts the probe according to the first embodiment. FIG. 3 is a partial cross-sectional view taken through A-A of FIG. 1.

A plurality of transducers 212 is arranged in the probe 210 according to the present embodiment. In the present embodiment, a direction in which the plurality of transducers 212 is arranged refers to the short-axis (X-axis) direction.

In the present embodiment, the plurality of transducers 212 transmits ultrasonic waves toward a region (subject) P where a blood vessel to be observed is present, and obtains ultrasound image data based on reflected waves of the ultrasonic waves.

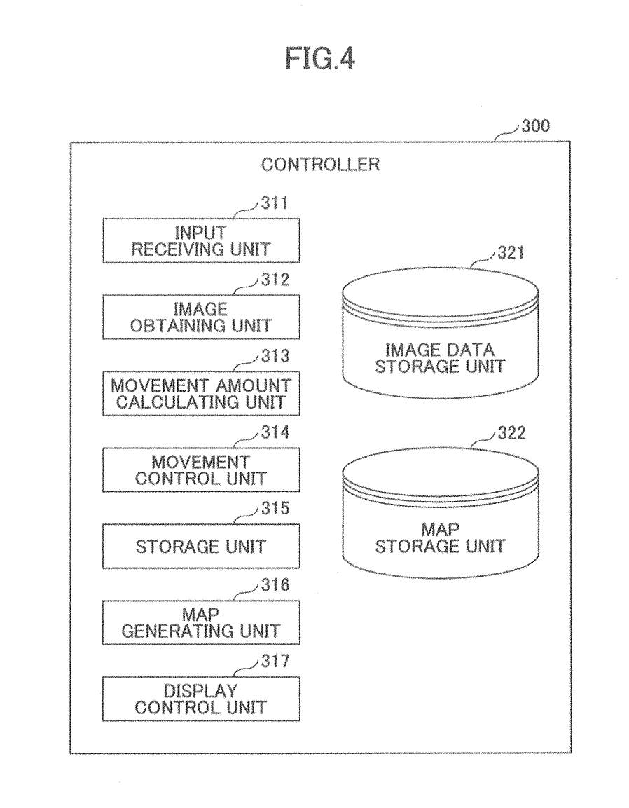

Next, referring to FIG. 4, functions of the controller 300 according to the present embodiment will be described. FIG. 4 is a drawing illustrating the functions of the controller according to the first embodiment. Each unit illustrated in FIG. 4 is implemented by causing the CPU 310 to read and execute programs stored in the memory 320.

The controller 300 according to the present embodiment includes an input receiving unit 311, an image obtaining unit 312, a movement amount calculating unit 313, a movement control unit 314, a storage unit 315, a map generating unit 316, and a display control unit 317. Further, the controller 300 according to the present embodiment includes an image data storage unit 321 and a map storage unit 322.

The input receiving unit 311 receives various inputs to the controller 300. The image obtaining unit 312 obtains ultrasound image data sequentially transmitted from the probe moving device 200 and received by the image obtaining unit 312.

Based on the ultrasound image data sequentially obtained by the image obtaining unit 312, the movement amount calculating unit 313 calculates an amount of movement in the X-axis direction of the probe 210. The amount of movement in the X-axis direction and the Y-axis direction may be associated with ultrasound image data used to calculate the amount of movement, and may be stored in the image data storage unit 321. A method of calculating an amount of movement by the movement amount calculating unit 313 will be described later in detail.

The movement control unit 314 outputs the amount of movement calculated by the movement amount calculating unit 313 to the probe driving device 360, and controls movement of the probe 210. Namely, the movement control unit 314 according to the present embodiment functions as a control unit that causes the probe 210 to move in the X-axis direction and the Y-axis direction.

The storage unit 315 stores ultrasound image data obtained by the image obtaining unit 312 in the image data storage unit 321 in association with information indicating a date and time when the ultrasound image data is obtained. Further, the storage unit 315 stores map data indicating a vascular access map created by the map generating unit 316 in the map storage unit 322.

Based on the ultrasound image data stored in the image data storage unit 321, the map generating unit 316 generates map data for displaying vascular access map.

Vascular access is a mechanism that circulates blood of a patient through a dialyzer when hemodialysis is performed, and is a collective term for an internal shunt, a superficial artery, a blood access catheter, and the like.

Further, map data generated by the map generating unit 316 is, for example, data in which each piece of ultrasound image data obtained by the image obtaining unit 312 is associated with information indicating a shape of a blood vessel to be observed, a depth of the blood vessel from the body surface, and a thickness of the blood vessel. The above information is calculated based on the obtained ultrasound image data. In other words, map data indicating a vascular access map generated by the map generating unit 316 is blood vessel map data indicating information related to the blood vessel of the patient. Namely, the map generating unit 316 according to the present embodiment functions as a generating unit that generates blood vessel map data. The map data indicating the vascular access map will be described later in detail.

Based on the map data generated by the map generating unit 316, the display control unit 317 causes the output device 350 to output the vascular access map. In other words, the display control unit 317 displays the vascular access map generated by the map generating unit 316 on the display of the controller 300.

The image data storage unit 321 stores ultrasound image data obtained by the image obtaining unit 312. The map storage unit 322 stores map data indicating a vascular access map generated by the map generating unit 316.

Further, the image data storage unit 321 and the map storage unit 322 may be provided in a predetermined memory area of the memory 320, for example, or may be provided in a device located outside of the controller 300.

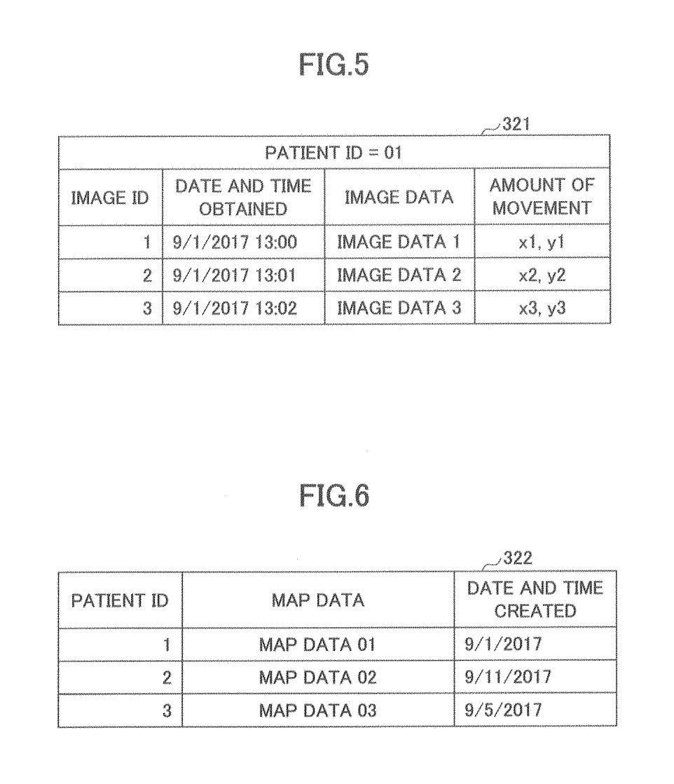

Referring to FIG. 5 and FIG. 6, the image data storage unit 321 and the map storage unit 322 will be described below.

In FIG. 5, in the image data storage unit 321 patient identification information for identifying each patient is stored in association with ultrasound image data sequentially obtained by the image obtaining unit 312 in accordance with the movement of the probe 210.

In the example of FIG. 5, a patient ID that is patient identification information for identifying a patient is associated with items “image ID,” “date and time obtained,” “image data,” and “amount of movement”.

Each value of the item “image ID” is identification information for identifying ultrasound image data obtained. Each value of the item “date and time obtained” is a date and time when ultrasound image data identified by a corresponding image ID is obtained by the image obtaining unit 312.

Each value of the item “image data” represents obtained ultrasound image data itself. Each value of the item “amount of movement” represents an amount of movement in the X-axis direction and in the Y-axis direction, which is obtained based on ultrasound image data identified by a corresponding image ID.

For example, in the example of FIG. 5, it can be seen that an amount of movement x1 is calculated based on ultrasound image data identified by an image ID “1” and obtained on Sep. 1, 2017 at 13:00 p.m.

FIG. 6 is a drawing illustrating an example of the map storage unit according to the first embodiment. Information stored in the map storage unit 322 according to the present embodiment includes items “patient ID,” “map data,” and “date and time created”. The item “patient ID” is associated with the other items.

Each value of the item “map data” is map data itself indicating a vascular access map created for a corresponding patient by the map generating unit 316. Each value of the item “date and time created” is a date and time when corresponding map data is created. Each of the values of the item “date and time created” may be obtained when the map generating unit 316 creates map data and may be stored in the map storage unit 322 in association with the created map data.

For example, in the example of FIG. 6, it can be seen that map data 01 indicating a vascular access map of a patient identified by a patient ID “01” is created on Sep. 1, 2017.

Next, a method of calculating an amount of movement by the movement amount calculating unit 313 according to the present embodiment will be described.

In initial ultrasound image data obtained at an initial position of the probe 210, the controller 300 according to the present embodiment receives specification of a blood vessel to be observed.

Upon the specification of the blood vessel being received, the movement amount calculating unit 313 calculates an amount of movement in the X-axis direction such that the specified blood vessel is located at the center of an ultrasound image indicated by the ultrasound image data obtained by the movement amount calculating unit 313.

In the present embodiment, the amount of movement is output to the probe moving device 200 as the amount of movement of the probe 210 in the X-axis direction and in the Y-axis direction.

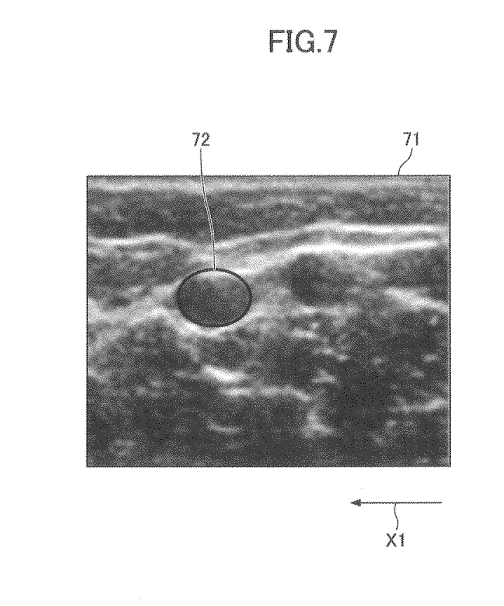

FIG. 7 illustrates an example of the ultrasound image data obtained by the image obtaining unit. In an ultrasound image 71 illustrated in FIG. 7, an image 72 represents an image of the specified blood vessel, which is to be observed. Further, the ultrasound image 71 is the next ultrasound image obtained after the first ultrasound image is obtained at the initial position.

In this case, in order for the image 72 to be located at the center of the ultrasound image 71, the ultrasound image 71 may be moved in the X-axis direction indicated by an arrow X1.

Thus, the movement amount calculating unit 313 may calculate an amount of movement in an X1 direction based on coordinates of the center point of the ultrasound image 71 and coordinates of the center point of the image 72. The coordinates as used herein refer to coordinates whose origin is taken as a given reference point in the ultrasound image 71.

Further, the amount of movement in the X1 direction calculated based on the ultrasound image 71 is stored in the image data storage unit 321 in association with the next ultrasound image obtained after the ultrasound image 71.

Further, in order to detect the image 72 representing the specified blood vessel from the ultrasound image 71, the movement amount calculating unit 313 according to the present embodiment may compare the initial ultrasound image obtained at the initial position with the ultrasound image 71, so as to detect, from the ultrasound image 71, an image that has a similar shape to that of the specified blood vessel of the initial ultrasound image obtained at the initial position.

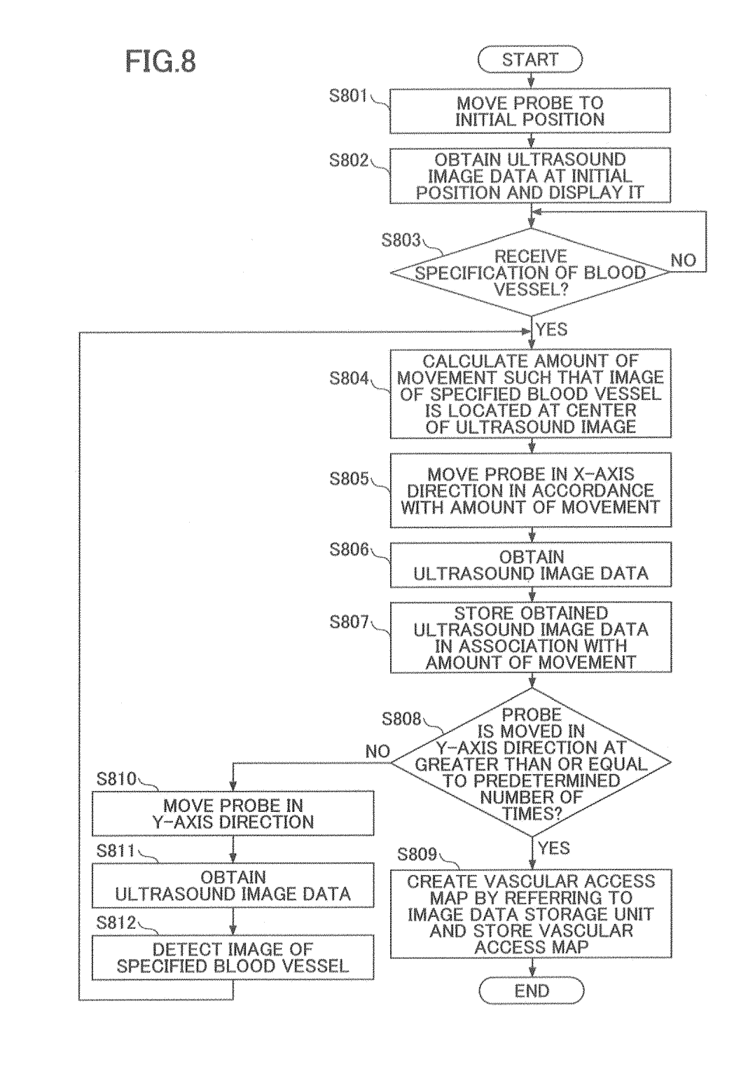

Next, referring to FIG. 8, a process performed by the controller 300 according to the present embodiment will be described. FIG. 8 is a flowchart illustrating the process performed by the controller 300 according to the first embodiment.

The controller 300 according to the present embodiment moves the probe 210 to the initial position in the probe moving device 200 (step S801). For example, information indicating the initial position of the probe 210 may be preliminarily set in the probe driving device 360. When the input receiving unit 311 receives an instruction to start obtaining ultrasound image data, the controller 300 may move the probe 210 to the preliminarily set initial position.

Next, the controller 300 causes the image obtaining unit 312 to obtain ultrasound image data at the initial position of the probe 210, and causes the display control unit 317 to display the obtained ultrasound image data on the display (step S802). The display may be, for example, the output device 350 of the controller 300, or may be an external display device other than the output device 350 of the controller 300.

Next, the controller 300 determines whether the input receiving unit 311 receives specification of a blood vessel on the display (step S803). In step S803, when no specification of a blood vessel is received, the controller 300 waits until specification of a blood vessel is received.

In step S803, when specification of a blood vessel is received, the controller 300 causes the movement amount calculating unit 313 to calculate an amount of movement in the X-axis direction such that an image of the specified blood vessel is located at the center of the obtained ultrasound image (step S804). The method of calculating an amount of movement by the movement amount calculating unit 313 is as described above. Information indicating the specified blood vessel may be kept in the memory 320.

Next, the controller 300 outputs the amount of movement calculated by the movement control unit 314 to the probe driving device 360, and moves the probe 210 in the X-axis direction (step S805). In other words, the controller 300 outputs the amount of movement to the probe driving device 360 and causes the probe driving device 360 to drive the moving mechanism 220. Accordingly, the moving mechanism 220 moves the probe 210 in accordance with the amount of movement.

Next, the controller 300 causes the image obtaining unit 312 to obtain ultrasound image data from the probe 210 (step S806).

Next, the controller 300 causes the storage unit 315 to store, in the image data storage unit 321, the ultrasound image data obtained in step S806 in association with the amount of movement in the X-axis direction and in the Y-axis direction (step S807). At this time, the ultrasound image data is provided with an image ID and is stored in the image data storage unit 321 together with information indicating a date and time when the ultrasound image data is obtained.

Next, the controller 300 determines whether the probe 210 is moved in the Y-axis direction at greater than or equal to a predetermined number of times (step S808).

In step S808, when the probe 210 is moved in the Y-axis direction at greater than or equal to the predetermined number of times, the controller 300 causes the map generating unit 316 to create map data indicating a vascular access map by referring to the image data storage unit 321, and stores the created map data in the map storage unit 322 (step S809). The controller 300 causes the process to end.

More specifically, based on a plurality of pieces of ultrasound image data and also based on an amount of movement in the X-axis direction and in the Y-axis direction associated with each of the plurality of pieces of ultrasound image data, the map generating unit 316 generates data indicating a plan view of the specified blood vessel. Further, based on an amount of movement in the Y-axis direction associated with each of the plurality of pieces of ultrasound image data, the map generating unit 316 generates data indicating a cross-sectional view of the specified blood vessel. Further, the map generating unit 316 associates the plurality of pieces of ultrasound image data, data indicating the plan view, and the data indicating the cross-sectional view with each other, and stores the associated data in the map storage unit 322 as map data indicating a vascular access map.

In step S808, if the probe 210 has not been moved in the Y-axis direction at greater than or equal to the predetermined number of times, the controller 300 moves the probe 210 in the Y-axis direction by a predetermined amount of movement (step S810). The predetermined amount of movement may be a preliminarily set amount of movement. For example, the predetermined amount of movement may be set in the probe driving device 360.

Next, the controller 300 causes the image obtaining unit 312 to obtain ultrasound image data from the probe 210 (step 3811). Next, from the ultrasound image data obtained in step S811, the controller 300 detects an image of a specified blood vessel (step S812), and causes the process to return to step S804.

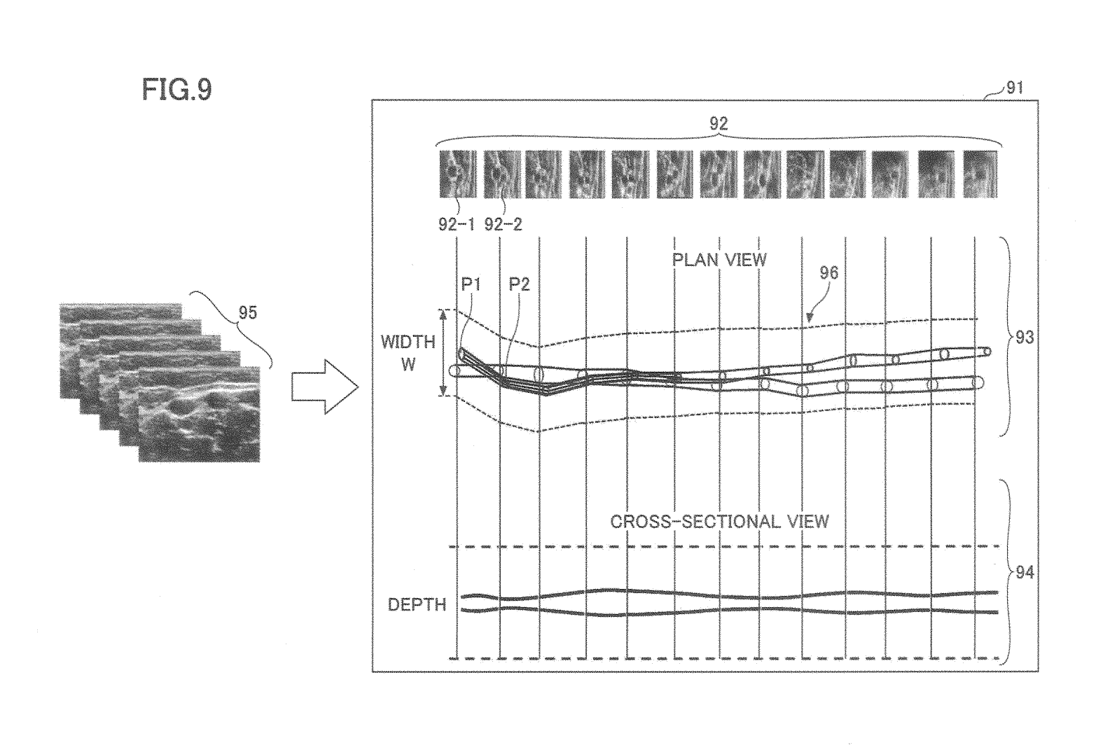

Referring to FIG. 9, the vascular access map generated by the controller 300 according to the present embodiment will be described below. FIG. 9 is a drawing illustrating an example of the vascular access map.

A vascular access map 91 illustrated in FIG. 9 is displayed by, for example, displaying map data generated by the map generating unit 316 on the display. The vascular access map 91 includes an ultrasound image display field 92, a plan view display field 93, and a cross-sectional view display field 94.

In the ultrasound image display field 92, a plurality of pieces of ultrasound image data included in an ultrasound image data group 95 stored in the image data storage unit 321 are displayed side-by-side. The plurality of pieces of ultrasound image data included in the ultrasound image data group 95 are ultrasound image data sequentially obtained each time the probe 210 is moved.

The plan view display field 93 displays a plan view of a blood vessel rendered based on an amount of movement in the X-axis direction and in the Y-axis direction, which is associated with each of the plurality of pieces of ultrasound image data included in the ultrasound image data group 95.

The cross-sectional view display field 94 displays a cross-sectional view of the blood vessel rendered based on the amount of movement in the Y-axis direction associated with each of the plurality of pieces of ultrasound image data included in the ultrasound image data group 95.

In the present embodiment, for example, a plot P1 in a plan view 96 is obtained based on an amount of movement in the X-axis direction and in the Y-axis direction, which is associated with ultrasound image data 92-1. In the plan view 96, a width W represents a width of the probe 210. The plot P1 indicates a position of the blood vessel within the width W at the time when the ultrasound image data 92-1 is obtained. Further, a plot P2 in the plan view 96 is obtained based on an amount of movement in the X-axis direction and in the Y-axis direction, which is associated with ultrasound image data 92-2.

In the present embodiment, the probe 210 is moved in the Y-axis direction by the predetermined amount of movement. Accordingly, the probe 210 is moved at equal intervals, and the plots are thus rendered at equal intervals in the plan view 96. The interval between each of the plots corresponds to the predetermined amount of movement in the Y-axis direction.

According to the present embodiment, when a blood vessel, which is to be observed, is specified, ultrasound image data is obtained by moving the probe 210 in the two directions of the X-axis direction and the Y-axis direction in such a manner that an image of the specified blood vessel is located at the center of the ultrasound image. Further, in the present embodiment, when the ultrasound image data is obtained, the probe 210 is moved in the Y-axis direction by the predetermined amount of movement and the above-described operation is performed.

Therefore, according to the present embodiment, by simply moving the probe 210 to the initial position, an ultrasound image data group in which an image of the blood vessel is located at the center of each ultrasound image can be obtained. Further, according to the present embodiment, map data indicating a vascular access map can be generated based on the ultrasound image data group and the amount of movement of the probe 210.

Therefore, in the present embodiment, techniques of scanning with an ultrasonic apparatus and of estimating a shape and a position of the blood vessel from an ultrasound image become unnecessary. Accordingly, a highly accurate vascular access map can be readily created.

### Second Embodiment

In the following, a second embodiment will be described with reference to the drawings. The second embodiment differs from the first embodiment in that a probe is provided with a mechanism of guiding a needle at a time of a puncture and that the probe is moved to a puncture position that is determined based on previous puncture positions. In the following description of the second embodiment, only the differences from the first embodiment will be described. Elements having the same functions and configurations as those in the first embodiment are referred to by the same numerals and a duplicate description thereof will be omitted.

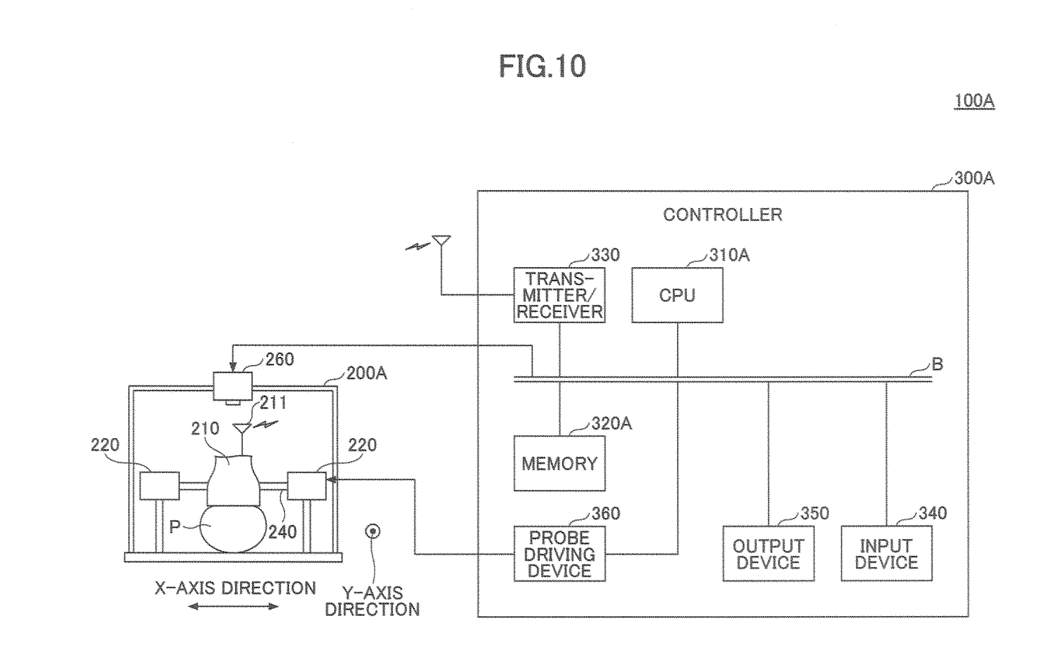

FIG. 10 is a drawing illustrating an example of an ultrasound diagnostic system according to the second embodiment. An ultrasound diagnostic system 100A according to the present embodiment includes a probe moving device 200A and a controller 300A.

The probe moving device 200A includes the probe 210, the moving mechanism 220, the rails 230, the rail 240, and a camera 260.

When a region of a patient, which is to be scanned by the probe 210, is placed between the rails 230, the probe moving device 200A causes the camera 260 to capture an image of the region so as to obtain image data. The probe moving device 200A transmits the image data to the controller 300A.

In the controller 300A according to the present embodiment, functions implemented by a CPU 310A and information stored in a memory 320A are different from those in the controller 300 according to the first embodiment.

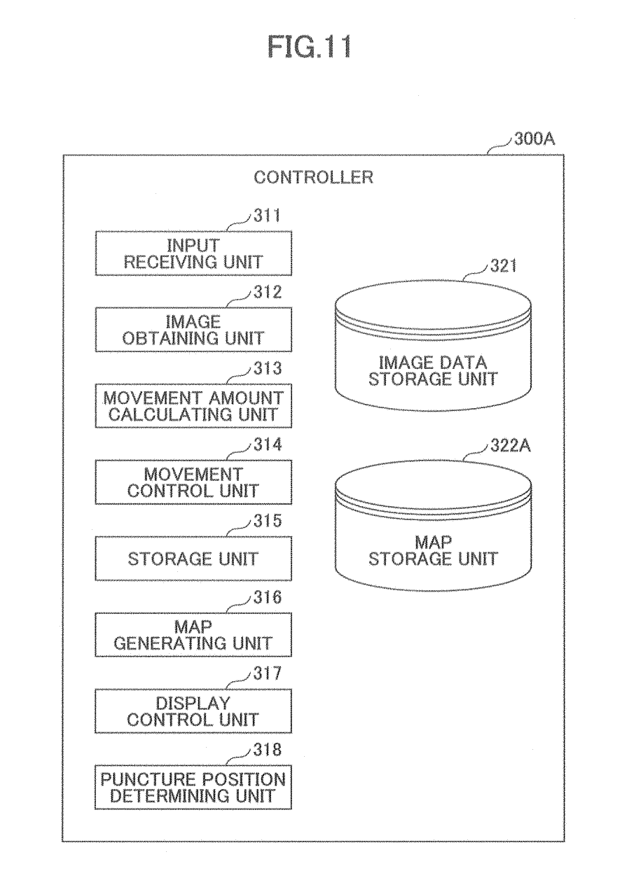

The functions of the controller 300A will be described below. FIG. 11 is a drawing illustrating the function of the controller according to the second embodiment. Each unit illustrated in FIG. 11 is implemented by causing the CPU 310A to read and execute programs stored in the memory 320A.

The controller 300A according to the present embodiment includes the input receiving unit 311, the image obtaining unit 312, the movement amount calculating unit 313, the movement control unit 314, the storage unit 315, the map generating unit 316, the display control unit 317, and a puncture position determining unit 318. Further, the controller 300A according to the present embodiment includes the image data storage unit 321 and a map storage unit 322A.

The puncture position determining unit 318 determines a puncture position based on position information representing puncture positions stored in map storage unit 322A for each patient and also based on date and time information representing dates and times when punctures are performed.

The map storage unit 322A stores map data for each patient in association with position information representing puncture positions and date and time information representing dates and times when punctures are performed.

The term puncture includes a puncture in which a needle is inserted into a blood vessel in order to remove blood from a patient's body and also a puncture in which a needle is inserted in a blood vessel in order to return blood into the patient's body.

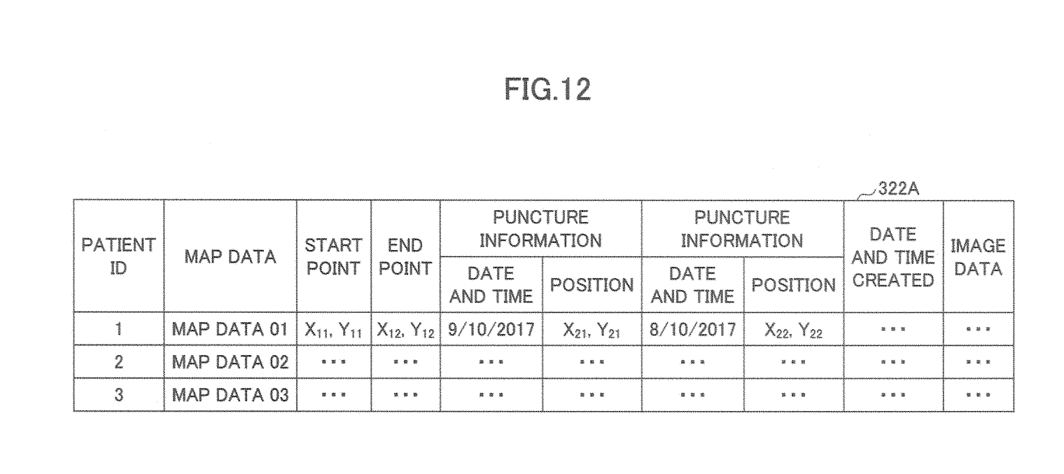

Referring to FIG. 12, the map storage unit 322A according to the present embodiment will be described below. FIG. 12 is a drawing illustrating an example of the map storage unit according to the second embodiment.

Information stored in the map storage unit 322A according to the present embodiment includes items “patient ID,” “map data,” “start point,” “end point,” “puncture information,” “date and time created,” and “image data”. The item “patient ID” is associated with the other items.

Each value of the item “start point” indicates a position at which the probe 210 starts scanning in an image of a patient's region captured by the camera 260. Each value of the item “end point” indicates a position at which the probe 210 ends scanning in an image of a patient's region captured by the camera 260. The values of the items “start point” and “end point” may be coordinate information. For example, the coordinate information may be coordinates of a position in an image of a patient's region indicated by the guide of the probe 210.

The values of the item “start point” and “end point” according to the present embodiment may be preliminarily specified in an image captured by the camera 260. Further, when a blood vessel, which is to be observed, is specified in image data captured by the camera 260, the values of the items “start point” and “end point” according to the present embodiment may be determined by the controller 300A so as to match the shape of the specified blood vessel.

The item “puncture information” is information in which the item “position information” and the item “date and time information” are associated with each other. Each value of the item “position information” represents a punctured position. Each value of the item “date and time information” represents a date and time when a puncture is performed.

The item “position information” may be coordinate information indicating a position of the guide of the probe 210 in the region of the patient. Further, the item “position information” may be an amount of movement in the X-axis direction and in the Y-axis direction after the probe 210 starts scanning. The item “puncture information” is added each time a puncture is performed.

The item camera 260 “image data” is image data captured by the camera 260.

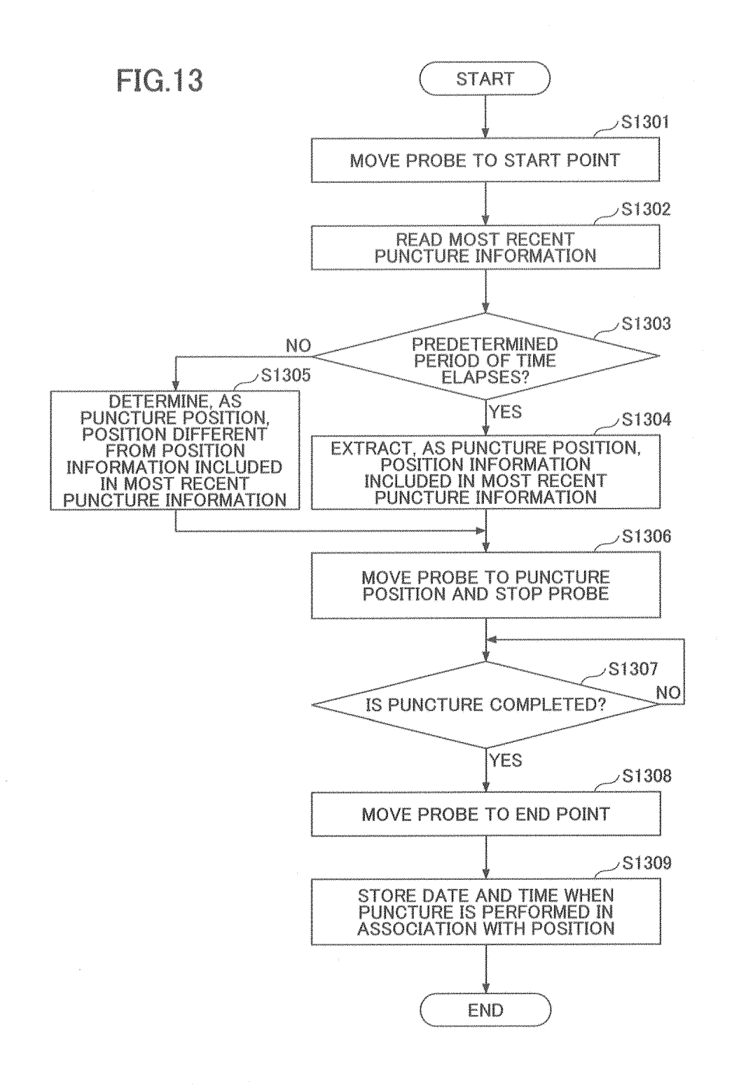

Next, referring to FIG. 13, a process performed by the controller 300A according to the present embodiment will be described. FIG. 13 is a flowchart illustrating the process performed by the controller according to the second embodiment.

When the input receiving unit 311 receives specification of a patient ID as well as a request for determining puncture positions, the controller 300A causes the movement control unit 314 to refer to a start point corresponding to the specified patient ID and to move the probe 210 to the start point (step S1301). In other words, when the controller 300A receives a specified patient ID, the controller 300A causes the movement control unit 314 to output a start point corresponding to the patient ID to the probe driving device 360. Then, the controller 300A causes the probe driving device 360 to drive the moving mechanism 220 so as to move the probe 210 to the start point.

Next, the controller 300A causes the puncture position determining unit 318 to read, from puncture information corresponding to the specified patient ID, the most recent puncture information whose date and time information is the latest (step S1302).

Next, based on the date and time indicated by the date and time information read in step S1302, the controller 300A causes the puncture position determining unit 318 to determine whether a predetermined period of time elapses (step S1303).

In step S1303, when the predetermined period of time elapses, the puncture position determining unit 318 extracts, as a puncture position, a position indicated by position information included in the most recent puncture information read in step S1302 (step S1304). The process proceeds to step 1306, which will be described later.

In step S1303, when the predetermined period of time does not elapse, the puncture position determining unit 318 determines, as a puncture position, a position different from the position indicated by the position information included in the most recent puncture information read in step 31302 (step S1305). The process proceeds to step 1306, which will be described later.

In the following, step S1305 will be described. When the predetermined period of time does not elapse, the puncture position determining unit 318 according to the present embodiment reads map data corresponding to the specified patient ID, for example.

Based on a plan view and a cross-sectional view of a specified blood vessel, the puncture position determining unit 318 obtains position information different from the position indicated by the position information included in the most recent puncture information read in step S1302, and determines the obtained position information as a puncture position.

In this case, the information indicating the puncture position is preferably an amount of movement in the X-axis direction and in the Y-axis direction of the probe 210.

Next, the controller 300A causes the movement control unit 314 to output the information indicating the puncture position to the probe driving device 360. The probe driving device 360 moves the probe 210 to the determined puncture position and stops the probe 210 (step S1306).

Next, the controller 300A determines whether the puncture is completed (step S1307). In the present embodiment, when the input receiving unit 311 receives indication that the puncture is completed, it is determined that the puncture is completed, for example.

In step S1307, when the puncture is not completed, the controller 300A waits until the puncture is completed. In step S1307, when the puncture is completed, the controller 300A causes the movement control unit 314 to move the probe 210 to an end point corresponding to the specified patient ID (step S1308).

Next, the controller 300A causes the storage unit 315 to store date and time information indicating the date and time when the puncture is performed and also store position information indicating the puncture position in the map storage unit 322A as new puncture information (step S1309). The controller 300A causes the process to ends.

In the present embodiment, for example, a date and time when a request for determining a puncture position is received or a date and time when completion of a puncture is indicated may be obtained as date and time information. In the example of FIG. 13, the probe 210 is moved to the end point after the puncture is completed. However, the present embodiment is not limited thereto, and the probe 210 may be moved to the start point after the puncture is completed.

According to the present embodiment, a next puncture position can be determined based on previous dates and times when punctures are performed and also based on previous puncture positions. According to the present embodiment, it is possible to save time and effort for a medical professional to refer to past patient records when determining a puncture position.

Further, according to the present embodiment, the probe 210 is moved to a determined puncture position with which the guide coincides. Accordingly, a medical professional can perform a puncture through the guide without the need to find a puncture position by him/herself.

Further, in the present embodiment, a puncture position may be specified on a vascular access map, and the probe 210 may be moved to the specified position, for example.

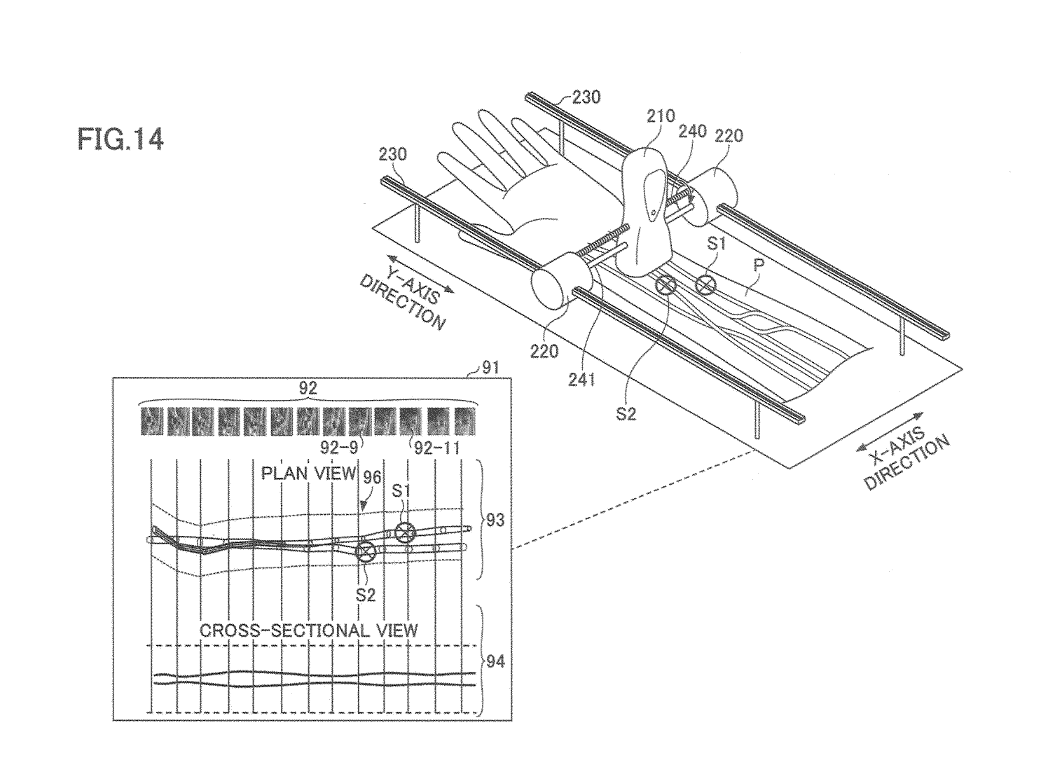

FIG. 14 is a drawing that depicts a method of specifying a puncture position on a vascular access map.

When the controller 300A according to the present embodiment receives a request for determining of puncture positions along with a patient ID, the controller 300A may display a vascular access map corresponding to the patient ID on the display and receive specification of puncture positions on the vascular access map.

In the example of FIG. 14, the vascular access map 91 is displayed, and a puncture position S1 for returning blood and a puncture position S2 for removing blood are specified on the plan view 96 of the blood vessel.

In this way, when the puncture positions are determined, the controller 300A causes the movement amount calculating unit 313 to refer to the image data storage unit 321 and to obtain an amount of movement corresponding to an ultrasound image 92-11 that includes the puncture position S1. Then, the controller 300A outputs the obtained amount of movement to the probe driving device 360. The probe driving device 360 drives the moving mechanism 220 based on the amount of movement and moves the probe 210 to the puncture position S1.

Also, the controller 300A causes the movement amount calculating unit 313 to refer to the image data storage unit 321 and to obtain an amount of movement corresponding to an ultrasound image 92-9 that includes puncture position S2. Then, the controller 300A outputs the obtained amount of movement to the probe driving device 360. The probe driving device 360 drives the moving mechanism 220 based on the amount of movement and moves the probe 210 to the puncture position S2.

Accordingly, in the present embodiment, puncture positions can be determined on a vascular access map. Further, according to the present embodiment, because the moving mechanism 220 conveys the probe 210 to the specified puncture positions, the probe 210 can be accurately moved to the specified puncture positions.

Further, in the present embodiment, on a plan view of a vascular access map displayed in order to determine puncture positions, marks indicating the most recent puncture positions indicated by puncture information stored in the map storage unit 322A may be displayed.

Further, in the present embodiment, IC tags may be embedded at positions where punctures are performed. In this case, the probe 210 may be provided with a tag reader. In the present embodiment, positions where the probe 210 has read signals from the IC tags may be used as position information indicating the most recent puncture positions.

Further, in the present embodiment, in a region of the patient, positions where punctures are performed may be marked, and an image of the region with the marked positions may be captured by the camera 260. The marked positions in the captured image may be kept as position information indicating positions of the most recent punctures. Further, in the present embodiment, marks may be placed on positions in an image captured by the camera 260 corresponding to positions where marks are displayed on a plan view of a vascular access map.

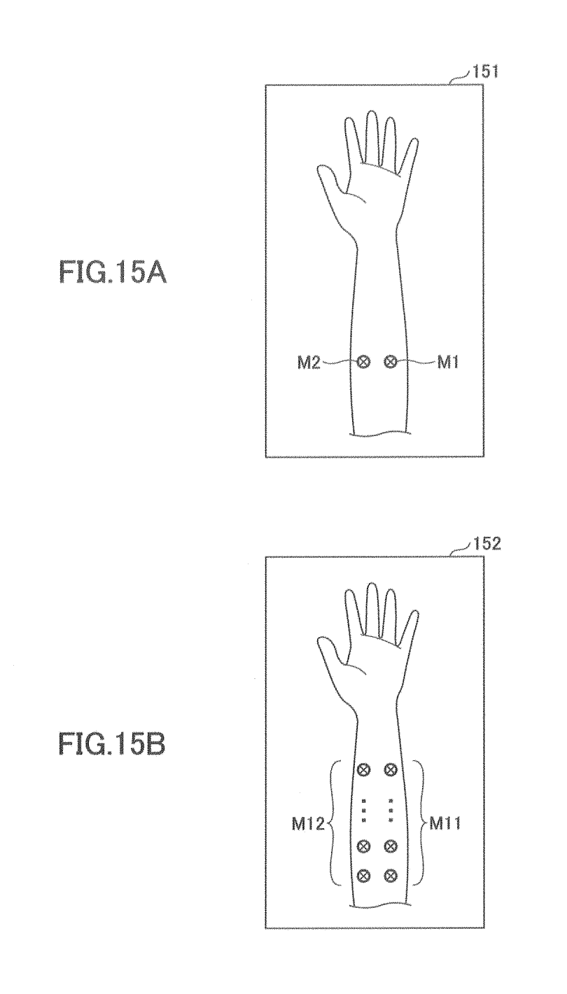

FIGS. 15A and 15B are drawings illustrating examples of images of a region of a patient. FIG. 15A illustrates an example of a captured image of the region of the patient where puncture positions are marked. FIG. 15B illustrates an example of a captured image in which marks are displayed at positions corresponding to positions of a vascular access map.

An image 151 illustrated in FIG. 15A is an image of the upper arm of the patient where puncture positions are marked. The image 151 includes a mark M1 indicating a position of the most recent puncture for returning blood, and also includes a mark M2 indicating a position of the most recent puncture for removing blood.

In an image 152 illustrated in FIG. 15B, marks corresponding to marks specified on a plan view of a vascular access map are displayed on the upper arm of the patient. In the image 152, marks M11 and marks M12 indicating positions of previous punctures indicated by puncture information stored in the map storage unit 322A are displayed.

### (Variations)

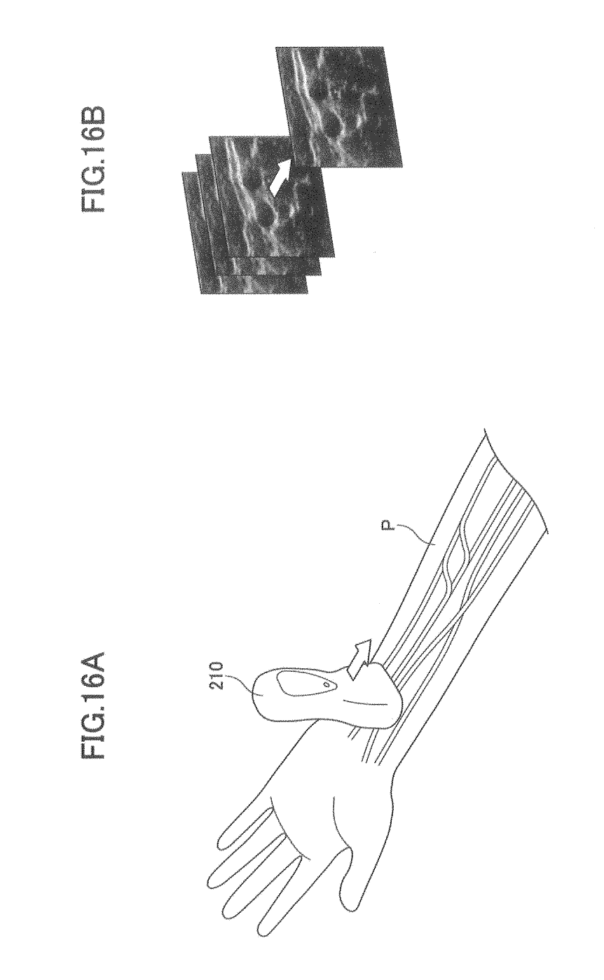

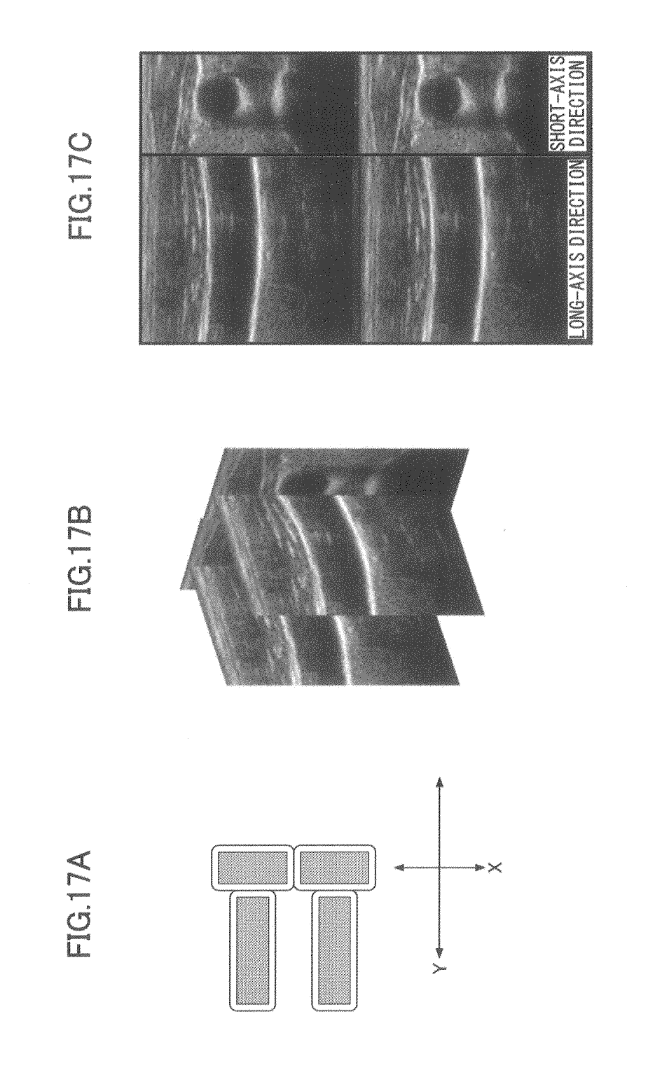

In the following, variations will be described with reference to FIGS. 16A through 17C. FIGS. 16A and 16B are drawings illustrating a first variation.

In the examples illustrated in FIGS. 16A and 16B, the probe 210 may be provided with an acceleration sensor such that an amount of movement in the X-axis direction and in the Y-axis direction of the probe 210 is obtained. The obtained amount of movement may be stored in the image data storage unit 321 in association with ultrasound image data obtained by the probe 210. A vascular access map may be created by referring to the image data storage unit 321.

FIGS. 17A through 17C are drawings illustrating a second variation. FIGS. 17A through 17C illustrate examples in which transducers of the probe are arranged in a T-shaped array.

FIG. 17A is a drawing illustrating shapes of the transducers. FIG. 17B is a drawing illustrating an example of an ultrasound image of a blood vessel obtained by the transducers arranged in the T-shaped array. FIG. 17C is a drawing illustrating an example of an ultrasound image when the blood vessel is scanned by the probe having the transducers arranged in the T-shaped array.

The examples of FIGS. 17A through 17C illustrate the transducers arranged in the short-axis direction and also in the long-axis direction perpendicular to the short-axis direction. The example of FIG. 17A illustrates the plurality of transducers arranged in the T-shaped array.

When the above-described transducers scan a blood vessel, an ultrasound image of the blood vessel as illustrated in FIG. 17B is obtained. Further, when the above-described transducers scan the body surface of the patient, an ultrasound image as illustrated in FIG. 17C is obtained.

As described, according to the present embodiment, positions at which punctures are to be performed can be determined based on previous dates and times when punctures are performed and also based on previous puncture positions.

According to at least one embodiment, a highly accurate vascular access map can be readily created.

Although the present invention has been described with reference to the embodiments, the present invention is not limited to the above-described embodiments, and variations and modifications may be suitably made without departing from the scope of the present invention.

What is claimed is:

1. An ultrasound diagnostic system comprising:
an ultrasonic probe having a plurality of transducers and configured to obtain a plurality of pieces of ultrasound image data of a subject, the plurality of transducers being arranged in a first direction and configured to transmit ultrasonic waves toward the subject and receive ultrasonic waves reflected from the subject;
a moving mechanism configured to move the ultrasonic probe in the first direction and in a second direction perpendicular to the first direction; and
a controller including a processor being configured to perform a process including,
causing the moving mechanism to move the ultrasonic probe based on a position of an image of a blood vessel to be observed of the subject in an ultrasound image indicated by a corresponding piece of the plurality of ultrasound image data, and
generating blood vessel map data indicating information related to the blood vessel to be observed by referring to an image data storage in which the plurality of pieces of ultrasound image data are stored in association with respective amounts of movement in the first direction and in the second direction.

2. The ultrasound diagnostic system according to claim 1, wherein the processor included in the controller is configured to perform the process further including:
causing a display device to display the ultrasound image indicated by the corresponding piece of ultrasound image data;
receiving specification of the blood vessel to be observed in the ultrasound image; and
calculating an amount of movement such that the image of the blood vessel to be observed is located at a center of the ultrasound image.

3. The ultrasound diagnostic according to claim 1, wherein the ultrasonic probe has a T-shaped transducer array that includes a plurality of transducers arranged in a line in the first direction and that includes a plurality of transducers arranged in the second direction.

4. The ultrasound diagnostic system according to claim 1, wherein the processor included in the controller is configured to perform the process further including,
causing the moving mechanism to move the ultrasonic probe in the second direction by a first amount of movement each time the ultrasonic probe obtains ultrasound image data.

5. The ultrasound diagnostic system according to claim 1, wherein the blood vessel map data is data for displaying, on the display device, a vascular access map that includes a plan view indicating a shape of the blood vessel to be observed and also includes an ultrasound image data group including the plurality of pieces of ultrasound image data obtained by the ultrasonic probe.

6. The ultrasound diagnostic system according to claim 5, wherein the processor included in the controller is configured to perform the process further including:
receiving specification of a puncture position for the subject in the vascular access map displayed on the display device; and
causing the moving mechanism to move the ultrasonic probe to the puncture position.

7. An ultrasound diagnostic system comprising:
an ultrasonic probe having a plurality of transducers and configured to obtain ultrasound image data of a subject, the plurality of transducers being arranged in a first direction and configured to transmit ultrasonic waves toward the subject and receive ultrasonic waves reflected from the subject;
a moving mechanism configured to move the ultrasonic probe along a body surface of the subject; and
a controller including a processor being configured to perform a process including,
determining a position of a puncture in an ultrasound image indicated by the ultrasound image data by referring to a storage in which position information indicating a previous position of a puncture performed for the subject is stored,
calculating an amount of movement of the ultrasonic probe based on the determined position, and
causing the moving mechanism to move the ultrasonic probe in accordance with the amount of movement.

8. The ultrasound diagnostic system according to claim 7, wherein the storage is further configured to store date and time information indicating a date and time when the puncture is performed, and the position of the puncture in the ultrasound image is determined based on the position information and the date and time information.

9. The ultrasound diagnostic system according to claim 7, comprising,
an imaging apparatus configured to capture an image of the subject,
wherein an initial position of the ultrasonic probe is specified in the image captured by the imaging apparatus.
